(19) United States
(12) Patent Application Publication
(10) Pub. No.: US 2022/0257685 A1
(43) Pub. Date: Aug. 18, 2022

(54) PLANT EXTRACT MIXTURE FOR USE IN THE PREVENTION AND/OR TREATMENT OF CHRONIC INFLAMMATORY BOWEL DISEASES

(71) Applicant: VALBIOTIS, PERIGNY (FR)

(72) Inventors: Sébastien PELTIER, FOURAS (FR); Pascal SIRVENT, CEYRAT (FR); Yolanda OTERO, CLERMONT FERRAND (FR)

(21) Appl. No.: 17/618,013

(22) PCT Filed: Jun. 11, 2020
(86) PCT No.: PCT/EP2020/066164

§ 371 Date: Dec. 15, 2021

(30) Foreign Application Priority Data
Jun. 11, 2019 (FR) FR1906165

(51) Int. Cl. A61K 36/287; A61K 36/28; A61K 36/45; A61K 31/7048; A61K 45/06; A61P 1/12

(52) U.S. Cl. CPC A61K 36/287; A61K 36/28; A61P 1/12; A61K 31/7048; A61K 45/06; A61K 36/45

(57) ABSTRACT

The invention relates to a composition comprising at least:
an extract of Chrysanthellum, and
an extract of artichoke, and
an extract of blueberry, and
an extract of at least one plant-derived raw material comprising oleuropein,
and optionally synthetic piperine and/or an extract of at least one plant-derived raw material comprising piperine,
   for use as a drug or nutritional product in the prevention and/or treatment of chronic inflammatory bowel disease in humans or animals.

### CROSS-REFERENCE TO RELATED APPLICATIONS

This application is a U.S. National Stage Application of PCT/EP2020/066164 assigned the international filing date of Jun. 11, 2020 and claiming the benefit of priority from French patent application FR1906165 filed Jun. 11, 2019, the disclosure of these applications is herein incorporated by reference.

### TECHNICAL FIELD

The present invention relates to a mixture of plant extracts for use as a drug or nutritional product in the prevention and/or treatment of chronic inflammatory bowel diseases (IBD).

### BACKGROUND

IBD, like Crohn's disease or ulcerative colitis, are conditions characterized by an inflammation of the lining of a part of the digestive tract related to an overactive digestive immune system. Symptoms are usually characterized by chronic abdominal pain, diarrhea and weight loss. These diseases progress through inflammatory attacks of extremely variable duration and frequency depending on the patient. These attacks alternate with phases of remission.

Most of the time, clinical management enables lasting control of the disease and a satisfactory quality of life other than during attacks, but there is no curative treatment. In addition, it is known that current treatments have side effects on bone density, weight gain and even exacerbate the intestinal disease (Asl Baakhtari S, McCombie A, Ten Bokkel Huinink S, Irving P, Siegel C A, Mulder R, et al. Observational Study of Perspectives of Inflammatory Bowel Disease Patients Concerning the Use of Corticosteroids. Dig Dis. 2018; 36(1):33-9. Epub 2017/09/04. doi: 10.1159/000478772. PubMed PMID: 28866661; Waljee A K, Wiitala W L, Govani S, Stidham R, Saini S, Hou J, et al. Corticosteroid Use and Complications in a US Inflammatory Bowel Disease Cohort. PLoS One. 2016; 11(6):e0158017. Epub 2016/06/24. doi: 10.1371/journal.pone.0158017. PubMed PMID: 27336296; PubMed Central PMCID: PMCPMC4918923).

The development of IBD is linked to several factors (genetic and environmental), but its etiology is not fully understood. Depending on the considered pathology, the extent and location of inflammation of the digestive tract may differ. This inflammation will lead to a change in transit time and disruption of the intestinal absorption function of water and nutrients. As a result, diarrhea is one of the most common symptoms in IBD patients, and may in particular be accompanied by the presence of blood in the stool when ulcerations are present. In addition, intestinal malabsorption often causes weight loss in affected patients. Although the physiopathological mechanisms leading to these physiological disturbances are not precisely known, they are the subject of much research, particularly on animal models recognized as mimicking this symptomatology. For example, mice treated with Dextran Sulfate Sodium (DSS) develop intestinal inflammation accompanied by an increase in the Disease Activity Index (DAI) taking into account the evaluation of 3 major symptoms found in the human pathology, namely weight loss, consistency of feces and bleeding, making these models particularly interesting for the identification of new therapeutic avenues (Randhawa P K, Singh K, Singh N, Jaggi A S. A review on chemical-induced inflammatory bowel disease models in rodents. Korean J Physiol Pharmacol. 2014 August; 18(4):279-88. doi: 10.4196/kjpp.2014.18.4.279. Epub 2014 Aug. 13. PubMed PMID: 25177159).

### SUMMARY OF THE INVENTION

The objective of the invention is to provide a product capable of acting on these symptoms and exhibiting greater efficacy than the reference drugs in the prevention and/or treatment of IBD.

To address this, the invention relates to the use of a composition comprising a mixture of several plant extracts.

In particular, the subject matter of the invention is a composition comprising at least:
an extract of Chrysanthellum, and
an extract of artichoke, and
an extract of blueberry, and
an extract of at least one plant-derived raw material comprising oleuropein,
and optionally synthetic piperine and/or an extract of at least one plant-derived raw material comprising piperine
   for use as a drug or nutritional product in the prevention and/or treatment of chronic inflammatory bowel diseases in humans or animals, in particular Crohn's disease and/or ulcerative colitis.

Such a composition is known for its effects on carbohydrate and/or lipid metabolism, but surprisingly, according to the invention, it is capable of greatly reducing the disease activity index in people or animals suffering from IBD. It can therefore be an effective drug for the prevention and/or treatment of IBD.

The invention is hereby described in detail.

### DETAILED DESCRIPTION OF THE INVENTION

### Definitions

“Analogs” (of a compound) within the meaning of the present invention means all compounds having a chemical structure similar to another compound, but different from it by a certain component. It may differ by one or more atoms, functional groups or substructures, which are replaced by other atoms, functional groups or substructures.

“Extract of a plant “X”” within the meaning of the invention means a set of molecules obtained from a single plant “X” by any suitable method. In particular, the following may be cited: aqueous extracts (obtained using an aqueous solvent), alcoholic extracts (obtained using an alcoholic solvent) or extracts using an organic solvent, or using a natural fatty substance or a mixture of natural fatty substances, in particular a plant-derived oil or a mixture of plant-derived oils.

“Mixture” within the meaning of the invention means the association of substances (extracts and/or molecules) in solid, liquid or gas form which may or may not interact chemically. The mixture of extracts according to the invention is obtained by any method known to those skilled in the art. It can be obtained by simply mixing the constituents.

“Plant” or “plant-derived raw material” within the meaning of the invention means the whole plant or part of a plant, including cell cultures, which has not yet undergone specific treatment and is intended to be used in the manufacturing of a plant preparation.

“Nutritional product” means all products having a nutritional and/or physiological effect; this in particular comprises food supplements, foods, dietetic products and the like. These products can in particular be administered by oral, gastric or venous route.

“Health product” means all the products having a beneficial effect on health, in prevention or in treatment, whether this effect is physiological or pharmacological, in particular drugs and pharmaceutical products. These products can in particular be administered by oral, gastric, venous or cutaneous route.

“Aqueous solvent” within the meaning of the invention means any solvent consisting entirely or partly of water. Thus, the following may be cited: water itself, hydro-alcoholic solvents in any proportion or even solvents consisting of water and of a compound such as glycerin or propylene glycol in any proportion. Among the alcoholic solvents, ethanol in particular may be cited.

### Compositions

In particular, the subject matter of the invention is a composition comprising at least:
an extract of Chrysanthellum, and
an extract of artichoke, and
an extract of blueberry, and
an extract of at least one plant-derived raw material comprising oleuropein, and
optionally synthetic piperine and/or an extract of at least one plant-derived raw material comprising piperine
   for use as a drug or nutritional product in the prevention and/or treatment of chronic inflammatory bowel disease in humans or animals.

In the present application, the singular or the plural will be used interchangeably to denote the useful compositions according to the invention.

The useful composition according to the invention therefore comprises a mixture of molecules comprising several extracts of plant-derived raw materials, including:
at least one extract of Chrysanthellum, preferably an extract of Chrysanthellum indicum, and
at least one extract of artichoke, preferably an extract chosen from extracts of Cynara cardunculus and extracts of Cyanara scolymus, and mixtures thereof, even more preferably an extract of Cynara scolymus, and
at least one extract of blueberry, preferably an extract of Vaccinium, in particular an extract chosen from extracts of Vaccinium Myrtillus, extracts of Vaccinium Angustifolium, extracts of Vaccinium Corymbosum, extracts of Vaccinium Ashei Reade, and mixtures thereof, even more preferably an extract of Vaccinium Myrtillus, and
an extract of at least one plant-derived raw material comprising oleuropein, preferably an extract chosen from extracts of olive tree (Oleo europeae), extracts of privet (Ligustrum), extracts of Argan tree, and mixtures thereof, even more preferably an extract of Olea europeae, and
preferably also synthetic piperine and/or an extract of at least one plant-derived raw material comprising piperine, preferably an extract chosen from extracts of Piper, extracts of Chalciporus piperatus, and mixtures thereof.

The plant extracts can be obtained by any suitable method, for example by a method comprising the following steps:
solid/liquid extraction
separation/pressing
filtration
evaporation
drying
optionally incorporating additives
homogenization
packaging

The extract of Chrysanthellum indicum is preferably an extract of whole plant or the aerial parts. It may in particular be a hydroalcoholic or aqueous extract or subcritical CO₂ or subcritical H₂O or associated with a heat treatment carried out by conventional heating or under microwave frequency or under ultrasound. The plant/extract ratio is preferably between 1/1 and 100/1, in particular between 1/1 and 25/1.

The composition according to the invention, when it is intended for humans, preferably comprises an amount of extract of Chrysanthellum indicum allowing the administration of at least 0.00001 g, in particular between 0.00001 g and 0.60 g, of extract of Chrysanthellum indicum per kg of body weight of the person to whom the composition is administered per day.

Preferably, the extract of Chrysanthellum indicum comprises at least one molecule chosen from apigenin-7-O-glucuronide, chrysanthellin A, chrysanthellin B, caffeic acid, luteolin, maritimetin, eryodictyol, isookanine, apigenin, luteolin-7-O-glucoside, maritimein, marein, eriodictyol-7-O-glucoside, flavomarein, apigenin-8-C-α-L-arabinoside-6-C-β-D-glucoside (shaftoside), apigenin-6,8-C-di-β-D-glucopyranoside (vicenin-2), or analogs thereof. Preferably, the extract comprises at least apigenin-7-O-glucuronide. Mention may also be made, for example, of analogs of apigenin-7-O-glucuronide such as apigenin-7-apioglucoside, apigenin-8-C-glucoside (vitexin), apigenin-6-C-glucoside (isovitexin), apigenin-7-O-neohesperidoside, apigenin-7-glucoside, apigenin-7-apioglucoside.

The extract of Cynara cardunculus or of Cyanara scolymus is preferably an extract of leaves or of roots. It may in particular be a hydroalcoholic or aqueous extract or subcritical CO₂ or subcritical H₂O or associated with a heat treatment carried out by conventional heating or under microwave frequency or under ultrasound. The plant/extract ratio is preferably between 1/1 and 100/1, in particular between 1/1 and 30/1.

The useful composition according to the invention, when it is intended for humans, preferably comprises an amount of extract of Cynara cardunculus or of Cyanara scolymus corresponding to a dose of at least 0.00001 g, in particular between 0.00001 g and 0.60 g of extract of Cynara cardunculus or of Cyanara scolymus per kg of body weight of the person to whom the composition is administered per day.

Preferably, the extract of Cynara cardunculus or of Cyanara scolymus comprises at least one molecule chosen from a dicaffeoylquinic acid, a sulfo-monocaffeoylquinic acid, luteolin, luteolin-7-0-glucoside, luteolin-7-O-glucuronide, apigenin-7-O-glucoside, cynaropicrin, or analogs thereof. Preferably, the extract comprises at least one dicaffeoylquinic acid.

The Vaccinium extract is preferably an extract of fruits or leaves. It may in particular be a hydroalcoholic or aqueous extract or subcritical CO₂ or subcritical H₂O or associated with a heat treatment carried out by conventional heating or under microwave frequency or under ultrasound. The plant/extract ratio is preferably between 1/1 and 200/1, in particular between 1/1 and 60/1.

The composition according to the invention, when it is intended for humans, preferably comprises an amount of extract of Vaccinium allowing the administration of at least 0.00001 g, in particular between 0.00001 g and 0.60 g, of extract of Vaccinium per kg of body weight of the person to whom the composition is administered per day.

Preferably, the extract of Vaccinium comprises at least one molecule chosen from a monocaffeoylquinic acid, delphinidin-3-galactoside, delphinidin-3-glucoside, cyanidin-3-galactoside, delphinidin-3-arabinoside, cyanidin-3-glucoside, petunidin-3-galactoside, cyanidin-3-arabinoside, petunidin-3-glucoside, peonidin-3-galactoside, petunidin-3-arabinoside, peonidin-3-glucoside, malvidin-3-galactoside, malvidin-3-glucoside, malvidin-3-arabinoside, or analogs thereof. Preferably, the extract comprises at least one monocaffeoylquinic acid.

The composition according to the invention, when it is intended for humans, preferably comprises an amount of piperine allowing the administration of at least 0.001 mg, in particular between 0.001 mg and 166 mg, of piperine per kg of body weight of the person to whom the composition is administered per day. If the piperine is contained in an extract of Piper, the mixture of the composition according to the invention comprises said extract. The extract of Piper is preferably an extract of Piper nigrum, of Piper aduncum and/or of Piper longum.

The extract of Piper is preferably an extract of fruits or leaves. It may in particular be a hydroalcoholic or aqueous extract or subcritical CO₂ or subcritical H₂O or associated with a heat treatment carried out by conventional heating or under microwave frequency or under ultrasound. The plant/extract ratio is preferably between 1/1 and 10,000/1, in particular between 1/1 and 200/1.

The extract preferably comprises at least 1% of piperine by weight relative to the total weight of the extract.

If the oleuropein is contained in an extract of Olea europaea, said extract is preferably an extract of leaves or fruits. It may in particular be a hydroalcoholic or aqueous extract or subcritical CO₂ or subcritical H₂O or associated with a heat treatment carried out by conventional heating or under microwave frequency or under ultrasound. The plant/extract ratio is preferably between 1/1 and 200/1, in particular between 1/1 and 60/1.

The composition according to the invention, when it is intended for humans, preferably comprises an amount of extract of Olea europaea allowing the administration of at least 0.00001 g, in particular between 0.00001 g and 0.60 g, of extract of Olea europaea per kg of body weight of the person to whom the composition is administered per day.

Preferably, the extract of Olea europaea comprises at least one hydroxytyrosol molecule or an analog, in addition to oleuropein.

Preferably, the composition according to the invention comprises at least the following molecules (these molecules being included in the extract(s) constituting the composition according to the invention):
at least one molecule chosen from apigenin-7-O-glucuronide, chrysanthellin A, chrysanthellin B, caffeic acid, luteolin, maritimetin, eryodictyol, isookanine, apigenin, luteolin-7-O-glucoside, maritimein, marein, eriodictyol-7-O-glucoside, flavomarein, apigenin-8-C-α-L-arabinoside-6-C-β-D-glucoside (shaftoside), apigenin-6,8-C-di-β-D-glucopyranoside (vicenin-2), or analogs thereof, preferably apigenin-7-O-glucuronide; and
at least one molecule chosen from a dicaffeoylquinic acid, a sulfo-monocaffeoylquinic acid, luteolin, luteolin-7-O-glucoside, luteolin-7-O-glucuronide, apigenin-7-O-glucoside, cynaropicrin, or analogs thereof, preferably a dicaffeoylquinic acid; and
at least one molecule chosen from a monocaffeoylquinic acid, delphinidin-3-galactoside, delphinidin-3-glucoside, cyanidin-3-galactoside, delphinidin-3-arabinoside, cyanidin-3-glucoside, petunidin-3-galactoside, cyanidin-3-arabinoside, petunidin-3-glucoside, peonidin-3-galactoside, petunidin-3-arabinoside, peonidin-3-glucoside, malvidin-3-galactoside, malvidin-3-glucoside, malvidin-3-arabinoside, or analogs thereof, preferably at least one monocaffeoylquinic acid; and
oleuropein, and
optionally preferably also verbascoside and/or luteolin-7-O-glucuronide, and
optionally piperine.

According to a particularly suitable variant, the mixture of extracts comprises at least one dicaffeoylquinic acid, verbascoside, apigenin-7-O-glucuronide, luteolin-7-o-glucuronide, a monocaffeoylquinic acid, oleuropein and optionally piperine.

Alternatively and equivalently, one or more or all of the molecules of the single extract may be replaced by synthetic or natural molecules from other plants.

The compositions according to the invention, in their different variants, can consist exclusively of the described elements (plant extracts), which together constitute an active ingredient, or else can also comprise at least one additional element (products, molecules, extracts, active ingredients, excipients, etc.) added in addition to plant extracts, said additional element being able to be chosen from:
the following vitamins: B1, B2, B3, B5, B6, B8, B9, B12 C, A, D, E, K1 and K2;
the following compounds: obeticholic acid, corosolic acid, polyunsaturated fatty acids of the omega 6 and/or omega 3 family, orotic acid, pangamic acid, para-aminobenzoic acid, amygdalin, beta-glucans, carnitine, dimethylglycine, imeglimin, isoflavones, L-arginine, oxytocin, pectin, pyridoxamine, resveratrol, viniferine, L-citrulline;
the following trace elements and minerals: arsenic, boron, calcium, copper, iron, fluorine, iodine, lithium, manganese, magnesium, molybdenum, nickel, phosphorus, selenium, vanadium, zinc;
the following non-essential micro-constituents: conjugated linolenic acid, lipoic acid, carotenoids, carnitine, choline, coenzyme Q10, phytosterols, polyphenols from the tannins and lignans family, taurine;
pre-biotics, for example fructooligosaccharides, inulins, galactooligosaccharides, pectins, beta-glucans, and xylooligosaccharides;
probiotics, for example Lactobacillus, Bifidobacteria, Akkermansia, Clostridium, Escherichia, Faecalibacteria, Bacteroides, Eubacteria;
anti-inflammatory drugs, corticosteroids (for example budesonide, betamethasone, prednisolone, and other corticosterone derivatives), aminosalicylated derivatives;
monoclonal antibodies, for example infliximab, adalimumab, vedolizumab; aminosalicylated derivatives, for example mesalazine;
yeasts, for example red yeast rice (Monascus purpureus);
mushrooms, for example maitake;
products derived from insects compatible with the food and pharmaceutical sector;
marijuana and hashish;
coating agents, for example hypromellose, microcrystalline cellulose, stearic acid, talc, sugar, shellac, povidone, beeswax;
flavors, for example natural blueberry flavor or natural strawberry flavor;
acidifiers such as malic acid;
anti-caking agents, for example silicon dioxide or magnesium stearate;
thickeners such as xanthan gum, colloidal silica, mono and diglycerides of fatty acids;
stabilizers such as calcium phosphate;
emulsifiers such as soy lecithin;
bulking agents such as corn starch;
excipients, for example microcrystalline cellulose, magnesium stearate or dicalcium phosphate.

The compositions according to the invention may also comprise one or more extracts of at least one of the following plant-derived raw materials and/or one or more molecules contained in at least one of the following plant-derived materials: Abelmoschus esculentus, Abies Alba, Abies balsamea, Abies sibirica, Acacia nilotica, Acacia senegal, Achillea millefollium, Achyranthes bidentata, Acmella oleracea, Actaea racemosa, Actinidia chinensis, Actinidia deliciosa, Adansonia digitata, Adiantum capillus-veneris, Aesculus hippocastanum, Afromomum melegueta, Agathosma betulina, Agathosma crenulata, Agathosma serratifolia, Agrimonia eupatoria, Ajuga reptans, Albizia julibrissin, Alchemilla vulgaris, Alliara petiolata, Allium ampeloprasum, Allium cepa, Allium sativum, Allium schoenoprasum, Allium ursinum, Alnus glutinosa, Aloe ferox, Aloe vera, Aloysia citriodora, Alpinia galanga, Alpinia hainanensis, Alpinia officinarum, Alpinia oxyphylla, Althaea officinalis, Ammi visnaga, Amorphophallus konjac, Ananas comosus, Andographis paniculata, Anemarrhena asphodeloides, Anethum graveolens, Angelica archangelica, Angelica dahurica, Angelica pubescens, Angelica sinensis, Antennaria diocia, Anthriscus cerefolium, Anthyllis vulneraria, Aphanizomenon flos-aquae Ralfs, Apium graveolens, Arachis hypogaea, Aralia elata, Arctium lappa, Arctium minus, Argania spinosa, Armorica rustanica, Artemisia dracunculus, Artemesia vulgaris, Ascophyllum nodosum, Aspalathus linearis, Asparagus officinalis, Astragalus membranaceus, Atractylodes lancea, Atractylodes macrocephala, Auracaria columnaris, Avena staiva, Ayahuasca, Baccharis genistelloides, Bacopa monnierri, Ballota nigra, Bambusa bambos, Bellis perennis, Berberis vulgaris, Beta vulgaris, Betula alleghaniensis, Betula pendula, Betula pubescens, Bixa orellana, Borago officnalis, Boswellia serrata, Brassica napus, Brassica nigra, Brassica oleracea, Brassica rapa, Bupleurum chinense, Calendula officinalis, Calluna vulgaris, Camellia sinsensis, Capsella bursa-pastoris, Capsicum annuum, Carex arenaria, Carica papaya, Carling acaulis, Carphephorus odoratissmus, Carpinus betulus, Carthamus tinctorius, Carum carvi, Cassia fistula, Castanea sativa, Centaurea centaurium, Centaurea cyanus, Centaurium erythraea, Centella asiatica, Cerasus vulgaris, Ceratonia silliqua, Chaenomelum nobile, Chlorella vulgaris, Chondrus crispus, Chrysanthellum indicum, Cichorium intybus, Cinchona officinalis, cinchona pubescens, Cinnamomum camphora, Cinnamomum cassia, Cinnamomum verum, Cistanche salsa, Cistus incanus, Citrus aurantium, Citrus limon, Citrus maxima, Citrus medico, Citrus myrtifolia, Citrus reticulata blanco, Citrus sinsensis, Citrus paradisi, Clinopodium vulgare, Cnicus benedictus, Cochlearia officinalis, Cocos nucifera, Codonopsis pilosula, Coffea canephora, Coix lacryma-jobi var. mayyuen Stapf, Cola acuminata, Cola ballayi cornu, Cola nitida, Combretum micranthum, Commiphora mukul, Conyza canadensis, Coriandrum sativum, Cornus officinalis, Corylus avellana, Corymbia citriodora, Crataegus laevigata, Craetegus monogyna, Crithmum maritimum, Crocus sativus, Cucumis melo, Cucurbita pepo, Cuminum cyminum, Cupressus sempervirens, Cuscuta chinensis, Cyamopsis tetragonoloba, Cyathula officinalis, Cyclanthera pedata, Cydonia oblonga, Cymbopogon martini, Cymbopogon nardus, Cymbopogon winterianus, Cynara cardunculus, Cyperus rotundus, Daucus carota, Dendranthema grandiflorum, Desmodium adscendens, Dimocarpus longan, Dioscorea oppostifolia, Dioscorea villosa, Diospyros kaki Thunb., Dunaliella saliena, Echinacea augustifolia, Echinacea pallida, Echinacea purpurea, Elaegnus rhamnoides, Alettaria cardamomum, Eleutherococcus senticosus, Elymus repens, Epiobium augustifolium, Epilobium parviflorum, Equisetum arvense, Erica cinerea, Erica tetralix, Eriobotrya japonica, Eriodictyon californicum, Erodium cicutarium, Eryngium campestre, Eschscholzia californica, Eucalyptus dives Schauer, Eucalyptus globulus, Eucalyptus radiata, Eucalyptus smithii F. Muell, Eucommia ulmoides, Eugenia uniflora, Eugenia jambolana, Euphrasia stricta D. Wolff, Euterpe oleracea, Fagopyrum esculentum Moench, Follopia japonica, Ferula assa-foetida, Ficus carica, Filipendula ulmaria, Foeniculum vulgare Mill., Forsythia suspensa, Fragaria dodonei Ard., Frangula purshiana Cooper, Fraxinus excelsior, Fraxinus ortus, Fucus serratus, Fucus vesiculosus, Fumaria officinalis, Galeopsis segetum Neck., Galium odotarum, Galium verum, Gardenia jasminoides J. Ellis, Gastrodia elata Blume, Gelidium corneum J. V. Lamouroux, Gentiana lutea, Geranium robertianum, Geum urbanum, Ginkgo biloba, Glycine max, Glycyrrhiza glabra, Glycyrrhiza uralensis, Gracilaria gracilis, Grindelia camporum Greene, Grindelia robusta Nutt., Grindelia squarrosa Dunal, Gymnema sylvestris, Haematococcus pluvialis, Hamamemis virginiana, Harpagophytum procumbens, Harpagophytum zeyheri Decne., Hedeoma pluegioides Pers., Helianthus annuus, Helienthus tuberosus, Helichrysum arenarium, Helichrysum stoechas, Herniara glabra, Hibiscus sabdariffa, Hieracium pilosella, Himanthalia elongata, Hordeum vulgare, Houttuynia cordata Thunb., Huperzia serrata, Hyssopus officinalis, Ilex paraguariensis A. St.-Hill, Illicum verum, Impatients balsamina, Inula britannica, Inula helenium, lasminum grandiflorum, lasmium officinale, Juniperus communis, Justicia adhatoda, Kavalama urens, Krameria lappacea, Lagerstroemia speciosa, Laminaria digitata, Laminaria hyperborea, Lamium album, Larix decidua, Larix occidentalis, Laurus nobilis, Lavandula augustofolia, Lavandula latifolia, Ledum palustre, Leonurus cardiaca, Lepidium meyenii Walp., Lepidium sativum, Lespedeza capitata, Levisticum officinale, Lindera aggregata, Linus usitatissimum, Liquidambar styraciflua, Lotus corniculatus, Lycium chinense, Lycium barbarum, Lycopersicon esculentum, Lycopodium clavatum, Lycopus europaeus, Lythrum salicaria, Macadamia ternifolia F. muell, Macrocystis pyrifera, Magnolia officinalis, Malpighia glabra, Malus pumila, Malus domestica, Malus sylvestris, Malva sylvestris, Mangifera indica, Maranta arundinacea, Marrubium vulgare, Marsdenia cundurango, Marsdenia sylvestris, Mastocarpus stellatus, Matricaria chamomi Ila, Medicago sativa, Melaleuca alternifolia, Melaleuca cajuputi Powell, Melaleuca leucadendra, Melaleuca quinquenrvia, Melaleuca viridiflora, Melilotus altissimus Thuill., Melilotus officinalis, Mentha arvensis, Mentha x piperita, Menyanthes trifoliata, Mesembryanthemum crystallinum, Monarda didyma, Morinda citrifolia, Morinda officinalis, Morus alba, Morus nigra, Murraya koenigii, Musa x paradisiaca, Myrciaria dubia, Myristica flagrans Houtt., Myroxylon balsamurn, Myrtus communis, Nardostachys jatamansi, Nasturtium officinale R. Br., Nelumbo nucifera Gaertn., Nepeta cataria, Nepeta tenuifolia Benth., Nigella sativa, Ocimurn basilicum, Oenothera biennis, Ononis spinosa, ophiopogon japonicus, Opuntia ficus-indica, Origanum cornpactum Benth., Origanum majorana, Origanum vulgare, Orthosiphon aristatus, Oryza sativa, Paeonia lactiflora, Paeonia x suffruticosa Andrews, Palmaria palmata, Panax ginseng, Panax quinquefolius, Panicum miliacium, Papaver rhoeas, Parietaria officinalis, Passiflora edulis Sims, Pastinaca sativa, Paullinia cupana Kunth, Pelargonium graveolens, Perilla frutescens, Persea americana, Persicaria bistorta, Persicaria maculosa Gray, Petroselinurn crispum, Peucadanurn ostruthium, Peumus boldus Molina, Phaseolus vulgaris, Phellodendron amurense, Photinia melancarpa, Phyllanthus emblica, Physalis alkekengi, Phymatolithon calcareum, Picea abies, Pimenta dioca, Pimenta racemosa, Pimpinella anisum, Pimpinella major, Pimpinella saxfraga, Pinus mugo Turra, Pinus pinaster Aiton, Pinus sylvestris, Pistacia lentiscus, Plantago arenaria, Plantago lanceolata, Plantago major, Plantago ovata, Platycodon grandiflorus, Plectranthus barbatus Andrews, Pogostemom cablin, Polygala senega, Polygala sibirica, Polygala tenuifolia Willd., Polygonum aviculare, Populus nigra, Populus tremula, Populus tremuloides, Porphyra umbilicalis, Portulaca oleracea, Potentilla erecta, Primula veris, Prunella vulgaris, Prunus africana, Prunus armeniaca, Ribes nigrum, Ribes uva-crispa, Rosa canina, Rosa gallica, Rosa moschata, Rosa rubiginosa, Rosmarinus officinalis, Rubus caesius, Rubus fruticosus, Rubus idaeus, Rumex actetosa, Rumex acetosella, Rumex crispus, Rumex patienta, Ruscus aculeatus, Sachharina japonica, Saccharina latissima, Salix alba, Salix fragilis, Salix pentandra, Salix purpurea, Salvia officinalis L., Salvia officinalis subsp. lavandulifolia Gams, Salvia sclarea, Sambucus nigra, Sanguisorba officinalis, Sanicula elata Buch.-Ham. Ex D. Don, Santalum album, Santolina chamaecyparissus, Saposhnikovia divaricata, Sargassum fusiforme, Satureja hortensis, Satureja montana, Saussurea costus, Scrophularia ningpoensis Helmsl., Scutellaria baicalensis Georgi, Secale cereale, Sedum acre, Sedum roseum, Senna alexandrina Mill., Senna obustifolia, Smilax cordifolia Humb. & Bonpl., Smilax glabra Roxb., Smilax officinalis Kunth, Smilax purhampuy Ruiz, Smilax purhampuy Ruiz, Smilax regelli Killip and C. V. Morton, Smilax vanillidora Apt, Solanum melongena, Solanum tuberosum, Solidago virgaurea, Sorbus aucuparia, Spatholobus suberctus Dunn., Spinacia oleracea, Spirulina major Kützing, Spirulina maxima Geitler, Spirulina platensis Geitler, Stavhys officinalis, Stemmacantha carthamoides Dittrich, Stypholobium japonicum, Syzgium aromaticum, Tagetes erecta, Tamarindus indica, Tanacetum parthemium, Terminalia chebula Retz., Theobroma cacao, Thymus saturejoides Coss., Thymus serpyllum, Thymus vulgaris, Thymus zygis, Tilia cordata Mill., Tilia platyphyllos Scop., Tilia tomentosa Moench, Tilia euopaea, Tribulus terrestris, Trichosanthes kirilowii Maxim., Trifolium arvense, Trifolium campestre Schreb., Trifolium pratense, Trifolium repens, Trigonella caerulea, Trigonella foenum-graecum, Tricitum aestivum, Tricitum durum Desf., Tricitum spelta L., Tricitum turgidum, Tropaeolum majus, Turnera diffusa Willd., Ulmus glabra Huds., Ulmus glabra Huds., Ulmus pumila, Ulmus rubra Muhl., Ulva lactuca, Uncaria gambir Roxb., Uncaria rhynchophylla Miq., Uncaria tomentosa DC., Undaria pinnatifida Suringar, Urtica dioca, Urtica urens, Vaccinium macrocarpon, Vaccinium oxycoccos, Vaccinium vitis-idae, Valeriana jatamansi Jones, Valeriana officinalis, Vanilla planifolia Jacks, Verbascum densiflorum Bertol., Verbascum thapsus, Verbena officinalis, Veronica officinalis, Viburnum opulus, Vigna angularis Ohwi & H. Ohashi, Vinca major, Vinca minor, Viola palustris, Viola tricolor, Vitex agnus-castus, Vitex trifolia, Vitis vinifera, Zea mays, Zingiber officinale Roscoe, Ziziphus jujuba Mill.

### Uses

The compositions according to the invention are intended to be administered to humans or animals by any administration means, preferably orally. They may be in the form of powder, gel, emulsion, or in liquid form, and in particular in the form of tablets, capsules, gelcaps, sticks, sachets, ampoules, droppers or in injectable form.

The compositions according to the invention can be used as nutrition products or health products, in particular as a drug.

When used in a person or animal with IBD, they lead to a reduction in the disease activity index taking into account 4 major components of the symptoms observed in humans: weight loss, bleeding and the texture of the feces.

This effect thus allows the composition according to the invention to be used in the prevention and/or treatment of chronic inflammatory bowel diseases in humans or animals, in particular in the prevention and/or treatment of chronic abdominal pain and/or diarrhea and/or weight loss from chronic inflammatory bowel disease. The composition according to the invention particularly may be used in the prevention and/or treatment of Crohn's disease and/or ulcerative colitis.

The invention is here illustrated by examples of extracts and compositions, as well as by test results demonstrating the effectiveness of the compositions according to the invention, these examples and tests not being limiting.

### EXAMPLES

### Example 1: Example of Dry Extract of Chrysanthellum indicum for Integration into a Composition According to the Invention

The aerial parts of the plant, fresh or dry, are subjected to mechanical grinding until a coarse powder is obtained. This powder is then subjected to a maceration step for 10 to 24 hours at room temperature in a 70/10 water/ethanol mixture, then the obtained whole is subjected to a continuous leaching at 50° C. in a percolator with a 70/10 water/ethanol mixture, the plant/extract ratio being 3/1. The obtained extract is then subjected to liquid/liquid washes using a non-polar organic solvent such as di- or tri-chloromethane. After concentration by low pressure evaporation at 35° C., a liquid is obtained that is lyophilized for 24 hours to give a beige powder soluble in a water/alcohol mixture. This powder (dry extract) can be used directly or else mixed in an appropriate solvent before use.

### Example 2: Example of Dry Extract of Vaccinium myrtillus for Integration into a Composition According to the Invention

The blueberry in powder form obtained from the fruits of Vaccinium myrtillus is subjected to a maceration stage for 10 to 24 hours at room temperature in a 30/50 water/ethanol mixture, then the obtained whole is subjected to continuous leaching at 50° C. in a percolator with a 30/50 water/ethanol mixture, the plant/extract ratio being 10/1. The obtained extract is then subjected to liquid/liquid washes using a non-polar organic solvent such as di- or tri-chloromethane. After concentration by low pressure evaporation at 35° C., a liquid is obtained that is lyophilized for 24 hours to give a violet-colored powder soluble in a water/alcohol mixture.

### Example 3: Example of Dry Extract of Cynara scolymus for Integration into a Composition According to the Invention

The artichoke in powder form obtained from the leaves of Cynara scolymus is subjected to a maceration stage for 10 to 24 hours at room temperature in water, then the obtained whole is subjected to continuous leaching at 50° C. in a percolator with water, the plant/extract ratio being 2/1. The obtained extract is then subjected to liquid/liquid washes using a non-polar organic solvent such as di- or tri-chloromethane. After concentration by low pressure evaporation at 35° C., a liquid is obtained that is lyophilized for 24 hours to give a beige powder soluble in water.

### Example 4: Example of Dry Extract of Piper nigrum for Integration into a Composition According to the Invention

The fruit part of the plant, fresh or dry, is subjected to mechanical grinding until a coarse powder is obtained. The molecules of interest, present in this coarse powder, are then extracted with a water/ethanol mixture. The obtained extract is then subjected to stages of evaporation of the solvent, concentration, recrystallization, filtration and washing. If necessary, these steps are repeated several times to optimize the extraction yield. After a final step of recrystallization and filtration in aqueous medium, the obtained precipitate is dried and recovered.

### Example 5: Example of Dry Extract of Olea europaea for Integration into a Composition According to the Invention

The whole and air-dried olive leaves are crushed at −80° C. using a knife mill to obtain a fine and homogeneous powder. The obtained powder is then subjected to a maceration step for 10 to 24 hours in a 70/30 water/ethanol mixture. The step is carried out in a closed system with nitrogen bubbling at room temperature, or under microwave power of 800 watts or under an ultrasound frequency of 20 kHz for 2×3 min. The obtained whole is then subjected to continuous leaching at 50° C. in a percolator with a 70/30 water/ethanol mixture, the plant/extract ratio being 10/1. The obtained extract is then subjected to liquid/liquid washes using a non-polar organic solvent such as di- or tri-chloromethane. After concentration by low pressure evaporation at 35° C., a liquid is obtained that is lyophilized for 24 hours to give a green powder soluble in a water/alcohol mixture.

### Example 6: Example of a Composition According to the Invention in the Form of Gelcaps

The composition of Example 6 is in the form of gelcaps that may be administered orally. It comprises, expressed as a percentage by weight, relative to the total weight of the composition, 37.0% of dry extract of Example 1, 37.0% of dry extract of Example 1, 3.7% of dry extract of Example 3, 0.004% of dry extract of Example 4, and 22.2% of dry extract of Example 5.

The composition for 3 gelcaps is shown in Table 1 below.

TABLE 1

| List of ingredients | For 3 gelcaps | |
|---|---|---|
| Dry extract of aerial parts of | 200 | mg |
| Chrysanthellum indicum | | |
| Dry extract of Cynara scolymus leaves | 200 | mg |
| Dry extract of Vaccinium myrtillus fruit | 20 | mg |
| Dry extract of Piper nigrum fruit | 0.02 | mg |
| Dry extract of Olea europaea leaves | 120 | mg |

### Example 7: Example of a Composition According to the Invention in the Form of Tablets

The composition of Example 7 is in the form of tablets that may be administered orally. It comprises, expressed as a percentage by weight, relative to the total weight of the composition, 22.0% dry extract of aerial parts of Chrysanthellum indicum, 22.0% dry extract of leaves of Cynara scolymus, 2.2% dry extract of fruit of Vaccinium myrtillus, 13.2% dry extract of leaves of Olea europaea, and 0.2% dry extract of fruit of Piper nigrum. In addition to the mixture, the composition also comprises zinc, vitamins B9, PP, B5, H, B12, D, B6, B2, B2 and chromium. It also comprises excipients, in particular dicalcium phosphate, microcrystalline cellulose and magnesium stearate.

The composition for 1 tablet is shown in Table 2 below.

TABLE 2

| | | Nutritional |
|---|---|---|
| | | Reference |
| | In mg for | Value for |
| List of ingredients | 1 tablet | 1 tablet |
| Dry extract of aerial parts of | 220.0 | — |
| Chrysanthellum indicum | | |
| Dry extract of Cynara scolymus leaves | 220.0 | — |
| Dry extract of Vaccinium myrtillus fruit | 22.0 | — |
| Dry extract of Olea europaea leaves | 132.0 | — |
| Dry extract of Piper nigrum fruit | 2.0 | — |
| Dicalcium phosphate | 198.0 | — |
| Microcrystalline cellulose | 153.44 | — |
| Magnesium Stearate | 20.0 | — |
| Zinc bisglycinate | 12.99 | 33% |
| Vitamin B9 - folic acid | 7.30 | 33% |
| Vitamin PP - nicotinamide | 5.30 | 33.1% |
| Vitamin B5 - calcium pantothenate | 2.24 | 33.3 |
| Vitamin H - biotin | 1.66 | 33.2% |
| Vitamin B12 | 0.83 | 33.2% |
| Vitamin D3 | 0.64 | 32% |
| Vitamin B6 - pyridoxine hydrochloride | 0.56 | 32.9% |
| Vitamin B1 - thiamine hydrochloride | 0.48 | 32.7% |
| Vitamin B2 - riboflavin | 0.45 | 32.1% |
| Chromium picolinate | 0.11 | 32.5% |

### Example 8: Example of a Composition According to the Invention in the Form of a Powder to be Reconstituted in Water, Packaged in the Form of Sticks

The composition of Example 8 is in the form of a powder to be reconstituted in water, packaged in the form of sticks that can be administered orally. It comprises, expressed as a percentage by weight, relative to the total weight of the composition, 37.0% dry extract of aerial parts of Chrysanthellum indicum (example 1), 37.0% dry extract of leaves of Cynara scolymus (example 2), 3.7% dry extract of fruit of Vaccinium myrtillus (example 3), 0.2% dry extract of fruit of Piper nigrum (example 4) and 22.2% dry extract of leaves of Olea europaea (example 5). The composition also comprises vitamin B12 and chromium. As a flavor, it comprises a natural strawberry flavor. As an anti-caking agent, it comprises silicon dioxide. This composition does not comprise an anti-caking agent and a thickener.

The composition of such a product is shown in Table 3 below.

TABLE 3

| | | | Nutritional |
|---|---|---|---|
| | | | Reference |
| | | | Value for |
| List of ingredients | For 3 sticks | | 3 sticks |
| Dry extract of | 120 | mg | — |
| Vaccinium myrtillus fruit | | | |
| Dry extract of aerial parts | 1200 | mg | — |
| of Chrysanthellum indicum | | | |
| Dry extract of Cynara scolymus leaves | 1200 | mg | — |
| Dry extract of Piper nigrum fruit | 6 | mg | |
| Dry extract of Olea europaea leaves | 720 | mg | — |
| Vitamin B3 (inositol hexanicotinate) | 2.5 | μg | 100% |
| Chromium (chromium picolinate) | 20 | μg | 50% |

### In Vivo Evaluation of the Effectiveness of the Composition

In vivo experiments in animals were carried out so as to evaluate the effectiveness of the composition according to the invention on the prevention and development of IBD. The model chosen was a mouse fed a diet rich in lipids and subjected to the addition of Dextran Sodium Sulfate (DSS, chemical inflammatory agent) in its drinking water for 7 days. This model is recognized to induce gut dysbiosis and inflammation similar to that found in humans in the context of IBD (Eichele D D, Kharbanda K K, World J Gastroenterol. 2017 Sep. 7; 23(33):6016-6029. doi: 10.3748/wjg.v23.133.6016; Lee J, Lee H, Kim T, Kim M, Park Y, Kim J, Park K et al. Plos One 2017. 12(11):e0187515. doi: 10.1371/journal.pone.0187515).

8-week-old C57BI/6 male mice were fed a high-fat diet for 5 weeks. During the final week, 2% DSS was added to the drinking water. One group of mice was also supplemented with the incorporation into this lipid-rich diet of a composition comprising a mixture of the extracts of Examples 1 to 5, in a Chrysanthellum indicum/Cynara scolymus/Vaccinium myrtillus/Piper nigrum/Olea europaea ratio of 1/1/0.1/0.00325/0.6. The dose of this composition was 2% during the first 4 weeks, then 2.7% during the last week concomitant with the DSS treatment. Two other groups received a dose of two standard drugs for the treatment of IBD (a corticosteroid and an anti-TNF-alpha antibody), before starting treatment with DSS.

During the inflammation induction period with DSS, the weight of the mice was recorded daily and fecal samples were taken to calculate the Disease Activity Index (DAI). This index takes into account three parameters: weight loss, texture of the feces and the presence of blood in the feces (see Table 4: Parameters for calculating the disease activity index):

TABLE 4

| Points | Weight loss | Texture of feces | Presence of blood |
|---|---|---|---|
| 0 | <1% | Normal | No blood |
| 1 | 1-5% | | |
| 2 | 5-10% | Soft and “sticky” | Blood detected with |
| | | | a blood culture |
| 3 | 10-15% | | |
| 4 | >15% | Diarrhea | Blood visible in feces |

Table 5 shows the DAI calculated for the different groups on days 1, 2 and 6 of the inflammation induction period (Table 5: DAI on days 1, 2 and 6 of the inflammation induction period. *p<0.05 vs anti-TNF-alpha antibody. ¥p<0.05 vs HFD+DSS).

TABLE 5

| Group | HFD + DSS | HFD + DSS + CX | HFD + DSS + Cortic | HFD + DSS + Ac |
|---|---|---|---|---|
| Day 1 | 1.5 ± 0.7 | 0 ± 0* | 1.5 ± 0.6 | 2.6 ± 0.6 |
| Day 2 | 2.7 ± 0.7 | 1.4 ± 0.5 | 2.7 ± 0.3 | 2.3 ± 0.6 |
| Day 6 | 8.9 ± 0.3 | 6.2 ± 1^{^{¥}} | 6.8 ± 1.9 | 6.4 ± 1.6 |

It is observed that composition X decreased the DAI throughout the duration of the treatment compared to the untreated group. Surprisingly, on day 1, the group treated with the composition according to the invention is the only one that has a DAI of zero, lower than that observed in the groups treated with the reference drugs. In addition, the composition according to the invention significantly reduced the DAI at the end of induction with DSS (day 6) compared to the untreated group (from 8.9 to 6.2), while the reference drugs did not reach the significance level.

Thus, the composition according to the invention, by acting on disease activity in this way, may be used in the treatment of IBD.

1. A composition comprising at least:
an extract of Chrysanthellum, and
an extract of artichoke, and
an extract of blueberry, and
an extract of at least one plant-derived raw material comprising oleuropein,
and optionally synthetic piperine and/or an extract of at least one plant-derived raw material comprising piperine,
for use as a drug or nutritional product in the prevention and/or treatment of chronic inflammatory bowel disease in humans or animals.

2. The composition for use thereof according to claim 1, characterized in that the extract of Chrysanthellum is an extract of Chrysanthellum indicum.

3. The composition for use thereof according to claim 1, characterized in that the extract of artichoke is chosen from extracts of Cyanara cardunculus, extracts of Cynara scolymus and mixtures thereof.

4. The composition for use thereof according to claim 1, characterized in that the extract of blueberry is chosen from extracts of Vaccinium Myrtillus, extracts of Vaccinium Angustifolium, extracts of Vaccinium Corymbosum, extracts of Vaccinium ashei Reade and mixtures thereof.

5. The composition for use thereof according to claim 1, characterized in that the extract of plant-derived raw material comprising oleuropein is chosen from extracts of Olea europeae, extracts of privet, extracts of Argan tree and mixtures thereof.

6. The composition for use thereof according to claim 1, characterized in that the extract of plant-derived raw material comprising piperine is chosen from extracts of Piper, extracts of Chalciporus piperatus and mixtures thereof.

7. The composition for use according to claim 1, in the prevention and/or treatment of Crohn's disease and/or ulcerative colitis.

8. The composition for use according to claim 1, to reduce the activity index of the disease taking into account 3 major components of the symptoms observed in humans: weight loss, bleeding and texture of the feces.

9. The composition for use according to claim 1, characterized in that it comprises at least the following molecules present in the extracts:
at least one molecule chosen from apigenin-7-O-glucuronide, chrysanthellin A, chrysanthellin B, caffeic acid, luteolin, maritimetin, eryodictyol, isookanine, apigenin, luteolin-7-O-glucoside, maritimein, marein, eriodictyol-7-O-glucoside, flavomarein, apigenin-8-C-α-L-arabinoside-6-C-β-D-glucoside (shaftoside), apigenin-6,8-C-di-β-D-glucopyranoside (vicenin-2), and
at least one molecule chosen from a dicaffeoylquinic acid, a sulfo-monocaffeoylquinic acid, luteolin, luteolin-7-0-glucoside, luteolin-7-O-glucuronide, apigenin-7-O-glucoside, cynaropicrin, and
at least one molecule chosen from a monocaffeoylquinic acid, delphinidin-3-galactoside, delphinidin-3-glucoside, cyanidin-3-galactoside, delphinidin-3-arabinoside, cyanidin-3-glucoside, petunidin-3-galactoside, cyanidin-3-arabinoside, petunidin-3-gluco side, peonidin-3-galactoside, petunidin-3-arabinoside, peonidin-3-glucoside, malvidin-3-galactoside, malvidin-3-glucoside, malvidin-3-arabinoside,
at least oleuropein and possibly hydroxytyrosol, and
optionally verbascoside and/or luteolin-7-O-glucuronide, and
optionally piperine.

10. The composition for use according to claim 1, characterized in that it comprises at least one dicaffeoylquinic acid, verbascoside, apigenin-7-O-glucuronide, luteolin-7-o-glucuronide, a monocaffeoylquinic acid, oleuropein and optionally piperine.

11. The composition for use according to claim 1, characterized in that it is exclusively made up of:
an extract of Chrysanthellum, and
an extract of artichoke, and
an extract of blueberry, and
an extract of at least one plant-derived raw material comprising oleuropein,
and optionally synthetic piperine and/or an extract of at least one plant-derived raw material comprising piperine.

12. The composition for use according to claim 1, characterized in that it also comprises at least one additional element added in addition to plant extracts, said additional element being chosen from:
the following vitamins: B1, B2, B3, B5, B6, B8, B9, B12 C, A, D, E, K1 and K2;
the following compounds: obeticholic acid, corosolic acid, polyunsaturated fatty acids of the omega 6 and/or omega 3 family, orotic acid, pangamic acid, para-aminobenzoic acid, amygdalin, beta-glucans, carnitine, dimethylglycine, imeglimin, isoflavones, L-arginine, oxytocin, pectin, pyridoxamine, resveratrol, viniferine, L-citrulline;
the following trace elements and minerals: arsenic, boron, calcium, copper, iron, fluorine, iodine, lithium, manganese, magnesium, molybdenum, nickel, phosphorus, selenium, vanadium, zinc;
the following non-essential micro-constituents: conjugated linolenic acid, lipoic acid, carotenoids, carnitine, choline, coenzyme Q10, phytosterols, polyphenols from the tannins and lignans family, taurine;
pre-biotics;
probiotics;
anti-inflammatory drugs;
corticosteroids;
aminosalicylated derivatives;
monoclonal antibodies;
yeasts;
mushrooms;
products derived from insects compatible with the food and pharmaceutical sector;
marijuana and hashish;
coating agents;
flavorings;
acidifiers;
anti-caking agents;
thickeners;
stabilizers;
emulsifiers;
bulking agents;
excipients.

13. The composition for use according to claim 1, for oral administration.

14. The composition for use according to claim 1, characterized in that it is in the form of powder, gel, emulsion, or in liquid form, and in particular in the form of tablets, capsules, gelcaps, sticks, sachets, ampoules, droppers or in injectable form.
